(19) United States
(12) Patent Application Publication
(10) Pub. No.: US 2022/0015613 A1
(43) Pub. Date: Jan. 20, 2022

(54) ENDOSCOPE APPARATUS

(71) Applicant: OLYMPUS CORPORATION, Tokyo (JP)

(72) Inventor: Tomoyuki IIZUKA, Tokyo (JP)

(73) Assignee: OLYMPUS CORPORATION, Tokyo (JP)

(21) Appl. No.: 17/492,205

(22) Filed: Oct. 1, 2021

Related U.S. Application Data
(63) Continuation of application No. PCT/JP2019/015023, filed on Apr. 4, 2019.

(51) Int. Cl. A61B 1/01; A61B 1/00; A61B 1/018

(52) U.S. Cl. CPC A61B 1/01; A61B 1/00098; A61B 1/00177; A61B 1/018; A61B 1/00096

(57) ABSTRACT

An endoscope apparatus can include distal end portion; a raising base accommodation portion; an insertion channel; a camera; a raising base; a guide wire abutting portion; a first guide portion; and a second guide portion, and relative to edges of an image on a monitor, the first guide portion guides the guide wire to a first position such that the guide wire appears in the image with the guide wire intersecting a first edge, and the second guide portion guides the guide wire to a second position such that the guide wire appears in the image with the guide wire intersecting a second edge.

### CROSS REFERENCE TO RELATED APPLICATION

This application is a continuation application of PCT/JP2019/015023 filed on Apr. 4, 2019, the entire contents of which are incorporated herein by this reference.

### BACKGROUND OF THE INVENTION

### 1. Field of the Invention

The present invention relates to an endoscope apparatus that is provided inside a distal end portion of an insertion portion and has a raising base (forceps elevator) that can be freely raised and lowered with rotation.

### 2. Description of the Related Art

In recent years, treatment of diseased parts in digestive tract systems, pancreaticobiliary duct systems, and the like has been carried out using endoscope apparatuses provided with so-called side-viewing endoscopes, in each of which an image pickup unit is provided in a side surface of a distal end portion of an insertion portion of the endoscope on a side of a distal end in a longitudinal axis direction (hereinafter, simply referred to as a side of a distal end).

Examples of the treatment of pancreaticobiliary duct systems and the like using such side-viewing endoscopes include therapeutic treatment of collecting gallstones present in choledochal ducts and the like with balloons and gripping treatment instruments as well as diagnostic treatment of imaging biliary ducts and pancreatic ducts with endoscopes.

Since pancreatic ducts, biliary ducts, hepatic ducts, or the like are significantly thin ducts, it is difficult to insert distal end portions of endoscope insertion portions directly into these ducts in treatment of the pancreatic ducts, the biliary ducts, the hepatic ducts, or the like with endoscopes.

Therefore, in general, a distal end portion of a side-viewing endoscope insertion portion is inserted up to a part near a duodenal papilla, a guide wire caused to be inserted into a treatment instrument insertion channel inside the insertion portion is then caused to project from a side surface opening formed in a side surface of the distal end portion of the insertion portion and is inserted into one of the aforementioned ducts in a roentgenoscopy, and a treatment instrument such as a catheter is selectively inserted into the pancreatic duct, the biliary duct, or the hepatic duct using the guide wire as a guide.

According to the method, it is possible to insert and pull out the treatment instrument into and from the pancreatic duct, the biliary duct, or the hepatic duct via the guide wire any number of times once the guide wire is inserted into the aforementioned duct, which is a thin duct.

Note that when the guide wire or the treatment instrument is inserted from the side surface opening portion into the aforementioned duct, the insertion is carried out by causing a raising base provided in a raising base accommodation portion that communicates with the side surface opening portion of the distal end portion and at a position facing an opening at a distal end of the treatment instrument insertion channel in a longitudinal axis direction (hereinafter, simply referred to as a distal end) and configured to be freely raised and lowered with rotation in a known manner to be raised.

A technique of pulling out the treatment instrument from the pancreatic duct, the biliary duct, or the hepatic duct, and then causing the raising base to further be raised to cause the guide wire to be pinched between the raising base and a guide wire abutting portion provided above the distal end opening of the treatment instrument insertion channel at a distal end rigid portion configuring the distal end portion, thereby fixing the position of the guide wire when the treatment instrument is pulled out from the aforementioned duct is known.

Specifically, Japanese Patent Application Laid-Open Publication No. 2018-171255 discloses a configuration in which a groove with a substantially V-shaped section is formed at a substantially central portion of a guide surface of a raising base configured to guide a guide wire or a treatment instrument in a plan view of the guide surface, and the guide wire is pinched and fixed while deviating in a shearing manner between a surface of a guide wire abutting portion at a first position and the groove in the raising base (hereinafter, the fixing of the guide wire performed at the first position will be referred to as center locking), by the raising base being caused to be raised in a state in which the guide wire is locked at the groove, that is, in a state in which the guide wire is locked at the central portion of the guide surface.

Moreover, a configuration in which the guide wire is more strongly pinched and fixed with a surface of an edge portion of the raising base on a side near an observation optical system and a groove formed at a second position of the guide wire abutting portion than with the center locking configuration, between the edge portion and the groove (hereinafter, the fixing of the guide wire performed at the second position in a state in which the guide wire is brought near the side surface opening portion on the side of the observation optical system will be referred to as side locking), by the raising base being caused to be raised in a state in which the guide wire is brought near the side surface opening portion on the side of the observation optical system of an image pickup unit provided in the side surface of the distal end portion of the insertion portion along with the side surface opening portion is disclosed.

These configurations have an advantage that the guide wire is not pulled out from the aforementioned duct together when the treatment instrument is pulled out since the position of the guide wire is fixed. There is also an advantage not only when the treatment instrument is pulled out but also when the treatment instrument is inserted into the aforementioned duct via the guide wire remaining in the aforementioned duct that unintentional entrance of the guide wire into a deeper part of the aforementioned duct and perforation of the aforementioned duct are prevented.

Incidentally, fixation of the guide wire is performed within a visual field range (image pickup range) of the observation optical system.

Specifically, a surgeon performs the aforementioned center locking and side locking while viewing a monitor image of a site to be examined on a monitor configured to output the image of the site to be examined picked up by the image pickup unit.

### SUMMARY

An endoscope apparatus according to an aspect of the present invention includes: a distal end portion provided on a side of a distal end of an insertion portion in a longitudinal axis direction; a raising base accommodation portion provided inside the distal end portion and including a side surface opening at a part of an outer peripheral surface of the distal end portion; an insertion channel provided inside the insertion portion and including an opening at a distal end in the longitudinal axis direction opened to face the raising base accommodation portion; an image pickup unit provided in the distal end portion and configured to pick up an image of a site to be examined on a side on which the side surface opening is formed; a raising base provided in the raising base accommodation portion to face the opening of the insertion channel and configured to be freely raised and lowered with rotation; a guide wire abutting portion provided inside the distal end portion and configured such that a guide wire led out from the opening of the insertion channel toward the side surface opening abuts on the guide wire abutting portion with raising of the raising base; a first guide portion provided in the raising base and configured to be able to guide the guide wire led out from the opening of the insertion channel to and pinch and fix the guide wire at a first position of the guide wire abutting portion in a state in which the raising base is raised; and a second guide portion provided in the raising base away from the first guide portion in a rotation shaft direction of the raising base and configured to be able to guide the guide wire led out from the opening of the insertion channel to and pinch and fix the guide wire at a second position of the guide wire abutting portion in the state in which the raising base is raised, in which relative to edges of the image of the site to be examined on a graphical user interface configured to output the image of the site to be examined picked up by the image pickup unit, the first guide portion guides the guide wire to the first position such that the guide wire appears in the image with the guide wire intersecting a first edge, and the second guide portion guides the guide wire to the second position such that the guide wire appears in the image with the guide wire intersecting a second edge that is different from the first edge.

An endoscope apparatus according to another aspect of the present invention includes: a distal end portion provided on a side of a distal end of an insertion portion in a longitudinal axis direction; a raising base accommodation portion provided inside the distal end portion and including a side surface opening at a part of an outer peripheral surface of the distal end portion; an insertion channel provided inside the insertion portion and including an opening at a distal end in the longitudinal axis direction opened to face the raising base accommodation portion; an image pickup unit provided in the distal end portion and configured to pick up an image of a site to be examined on a side on which the side surface opening is formed; a raising base provided in the raising base accommodation portion to face the opening of the insertion channel and configured to be freely raised and lowered with rotation; a guide wire abutting portion provided inside the distal end portion and configured such that a guide wire led out from the opening of the insertion channel toward the side surface opening abuts on the guide wire abutting portion with raising of the raising base; a first guide portion provided in the raising base and configured to be able to guide the guide wire led out from the opening of the insertion channel to and pinch and fix the guide wire at a first position of the guide wire abutting portion in a state in which the raising base is raised; and a second guide portion provided in the raising base away from the first guide portion in a rotation shaft direction of the raising base and configured to be able to guide the guide wire led out from the opening of the insertion channel to and pinch and fix the guide wire at a second position of the guide wire abutting portion in the state in which the raising base is raised, in which in the state in which the raising base is raised, the first guide portion is disposed within an image pickup range of the image pickup unit, and the second guide portion is disposed outside the image pickup range.

Also, an endoscope apparatus according to another aspect of the present invention includes: a distal end portion provided on a side of a distal end of an insertion portion in a longitudinal axis direction; a raising base accommodation portion provided inside the distal end portion and including a side surface opening at a part of an outer peripheral surface of the distal end portion; an insertion channel provided inside the insertion portion and including an opening at a distal end in the longitudinal axis direction opened to face the raising base accommodation portion; an image pickup unit provided in the distal end portion and configured to pick up an image of a site to be examined on a side on which the side surface opening is formed; a raising base provided in the raising base accommodation portion to face the opening of the insertion channel and configured to be freely raised and lowered with rotation; a first guide portion provided in the raising base and configured to be able to guide, to a first position, a treatment instrument led out from the opening of the insertion channel; and a second guide portion provided in the raising base at a position different from a position of the first guide portion and configured to be able to guide, to a second position, the treatment instrument led out from the opening of the insertion channel, in which when the raising base is rotated and is in a first state, relative to edges of the image of the site to be examined on a graphical user interface configured to output the image of the site to be examined picked up by the image pickup unit, the first guide portion guides the treatment instrument to the first position such that the treatment instrument appears in the image with the treatment instrument intersecting a first edge, and the second guide portion guides the treatment instrument to the second position such that the treatment instrument appears in the image with the treatment instrument intersecting a second edge that is different from the first edge.

### BRIEF DESCRIPTION OF THE DRAWINGS

FIG. 1 is a perspective view illustrating an appearance of an endoscope apparatus according to a first embodiment;

FIG. 2 is an enlarged perspective view on a side of a distal end of an insertion portion surrounded by a line II in FIG. 1;

FIG. 3 is a perspective view illustrating a state in which a raising base in FIG. 2 is raised;

FIG. 4 is a perspective view illustrating a state in which center locking of a guide wire has been achieved by the raising base in FIG. 3;

FIG. 5 is a perspective view illustrating a state in which side locking of the guide wire has been achieved by the raising base in FIG. 3;

FIG. 6 is a top view of a distal end portion in FIG. 2 when seen in a VI direction in FIG. 2;

FIG. 7 is a cross-sectional view of the distal end portion along a line VII-VII in FIG. 6;

FIG. 8 is a cross-sectional view of the distal end portion along a line VIII-VIII in FIG. 6;

FIG. 9 is a perspective view illustrating an example in which a first guide portion formed in a guide surface of the raising base in FIG. 2 is configured with a V-shaped groove;

FIG. 10 is a perspective view illustrating a modification in which the first guide portion of the raising base in FIG. 2 is formed at a ridge line of the other end of the raising base;

FIG. 11 is a partial cross-sectional view illustrating a modification in which a second guide portion of the raising base in FIG. 2 is formed into a curved surface and a second position of a guide wire abutting portion is formed into a plane;

FIG. 12 is a partial cross-sectional view illustrating a modification in which the second guide portion of the raising base in FIG. 2 is formed into a plane and the second position of the guide wire abutting portion is formed into a curved surface;

FIG. 13 is a partial cross-sectional view illustrating a modification in which the second guide portion of the raising base in FIG. 2 is formed into a curved surface and the second position of the guide wire abutting portion is formed into a curved surface;

FIG. 14 is a diagram illustrating an example in which the guide wire appears in a monitor image displayed on a monitor in FIG. 1 in the case of the center locking and in the case of the side locking;

FIG. 15 is a diagram illustrating a configuration in which a raising base appears in a monitor image of a monitor in an endoscope apparatus according to a second embodiment; and

FIG. 16 is a diagram illustrating a modification in which guide wires are displayed in further proximity to each other in monitor images in a case of center locking and in a case of side locking as compared with FIG. 15.

### DETAILED DESCRIPTION OF THE PREFERRED EMBODIMENTS

Hereinafter, embodiments of the present invention will be described with reference to the drawings.

### First Embodiment

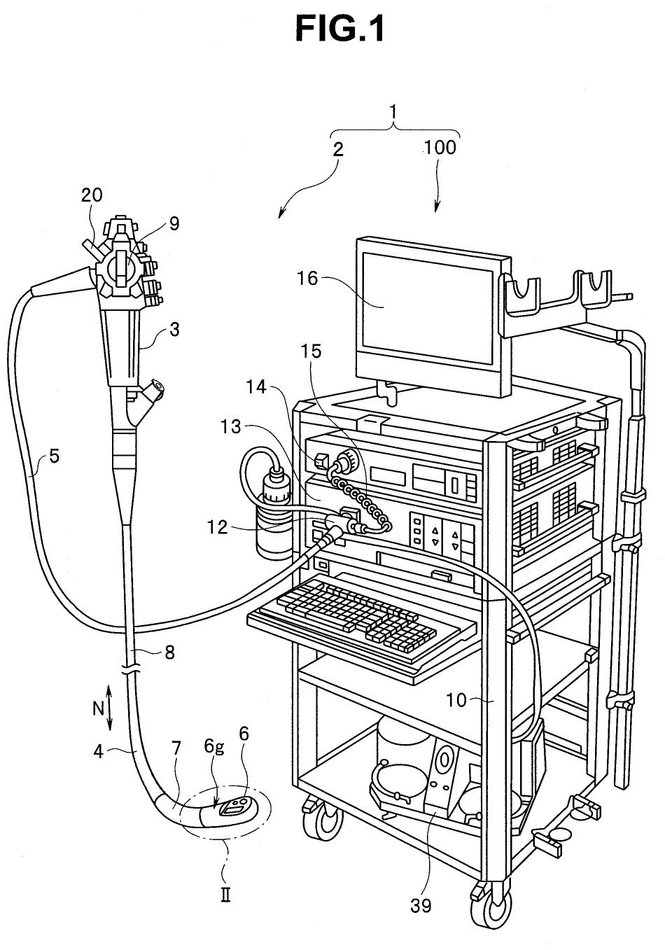

FIG. 1 is a perspective view illustrating an appearance of an endoscope apparatus according to the present embodiment.

As illustrated in FIG. 1, an endoscope apparatus 1 is configured with an endoscope 2 and a peripheral device 100.

The endoscope 2 has main components configured with an insertion portion 4 configured to be inserted into a subject, an operation portion 3 continuously provided on a side of a proximal end of the insertion portion 4 in a longitudinal axis direction N (hereinafter, simply referred to as a side of a proximal end), a universal cord 5 extending from the operation portion 3, and a connector 12 provided at an extending end of the universal cord 5.

The peripheral device 100 includes a light source device 13, a video processor 14, a connection cable 15 configured to electrically connect the light source device 13 to the video processor 14, and a monitor 16 configured to output an image G (see FIG. 14) of a site to be examined on a screen that is a graphical user interface, which are disposed on a stand 10.

Also, the endoscope 2 and the peripheral device 100 with such configurations are connected to each other with the connector 12.

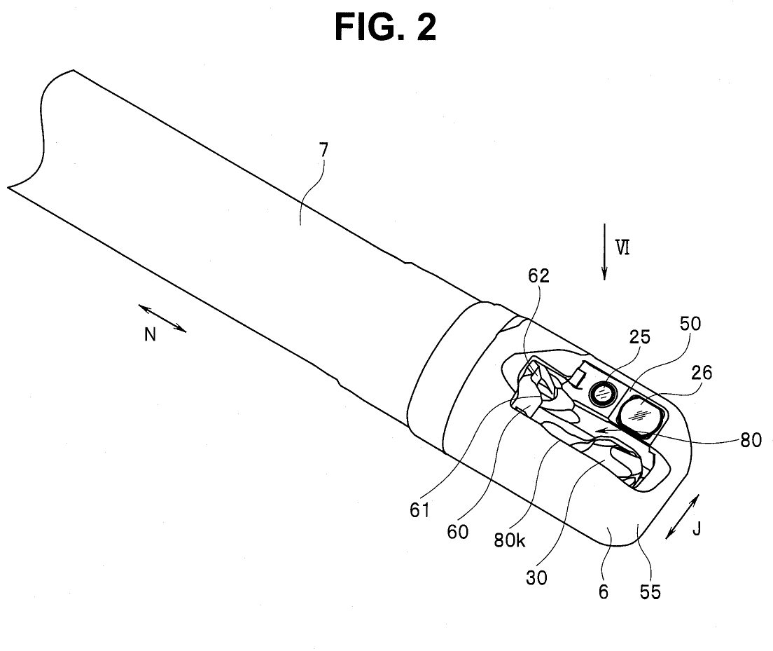

The operation portion 3 of the endoscope 2 is provided with a bending operation knob 9 and a treatment instrument raising base operation knob 20 provided inside a distal end portion 6 and configured to perform raising operation of a raising base 30 (see FIG. 2), which will be described later.

The insertion portion 4 of the endoscope 2 is configured with the distal end portion 6 located on a side of a distal end of the insertion portion 4, a bending portion 7 continuously provided at a proximal end of the distal end portion 6 in the longitudinal axis direction N (hereinafter, simply referred to as a proximal end), and a flexible tube portion 8 continuously provided at a proximal end of the bending portion 7.

The bending portion 7 is operated to bend by the bending operation knob 9 and is provided between the distal end portion 6 and the flexible tube portion 8 in the longitudinal axis direction N.

Next, a configuration related to the raising base 30 operated by the treatment instrument raising base operation knob 20 will be described using FIGS. 2 to 14.

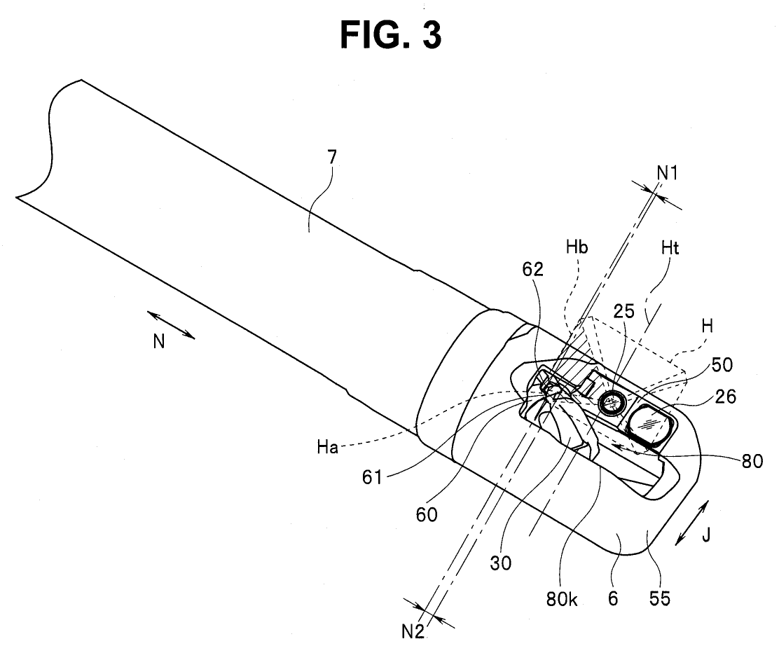

FIG. 2 is an enlarged perspective view on the side of the distal end of the insertion portion surrounded by the line II in FIG. 1, and FIG. 3 is a perspective view illustrating a state in which the raising base in FIG. 2 is raised.

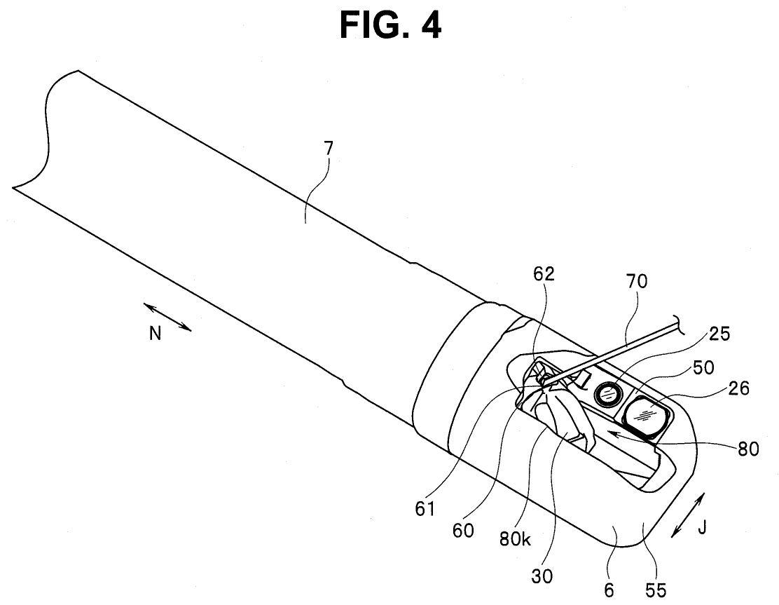

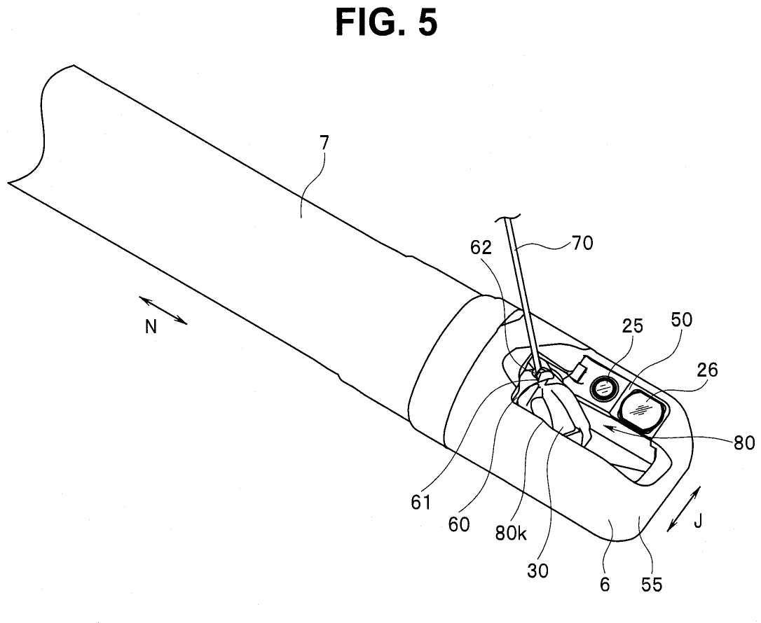

FIG. 4 is a perspective view illustrating a state in which center locking of the guide wire has been ben achieved by the raising base in FIG. 3, and FIG. 5 is a perspective view illustrating a state in which side locking of the guide wire has been achieved by the raising base in FIG. 3.

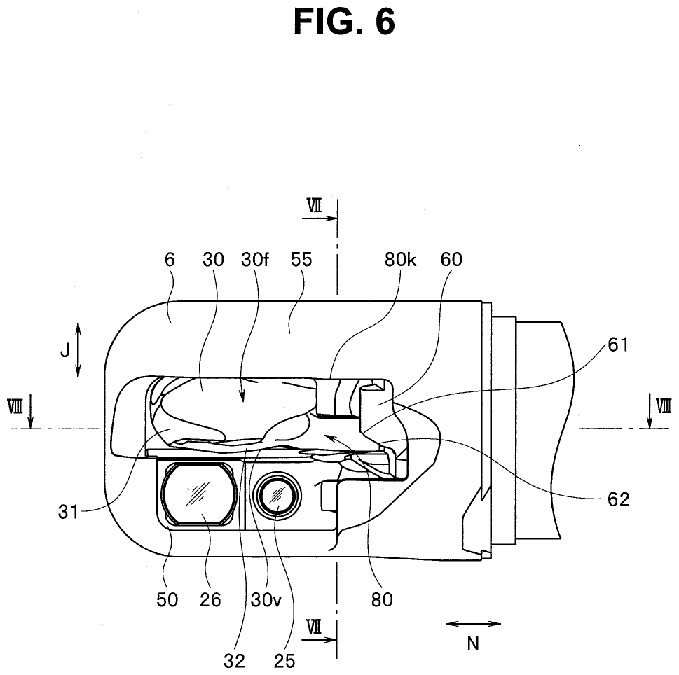

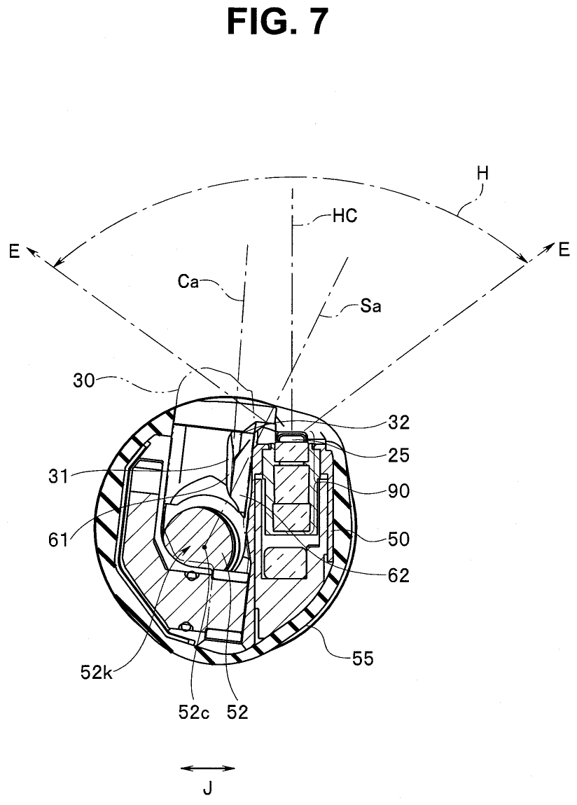

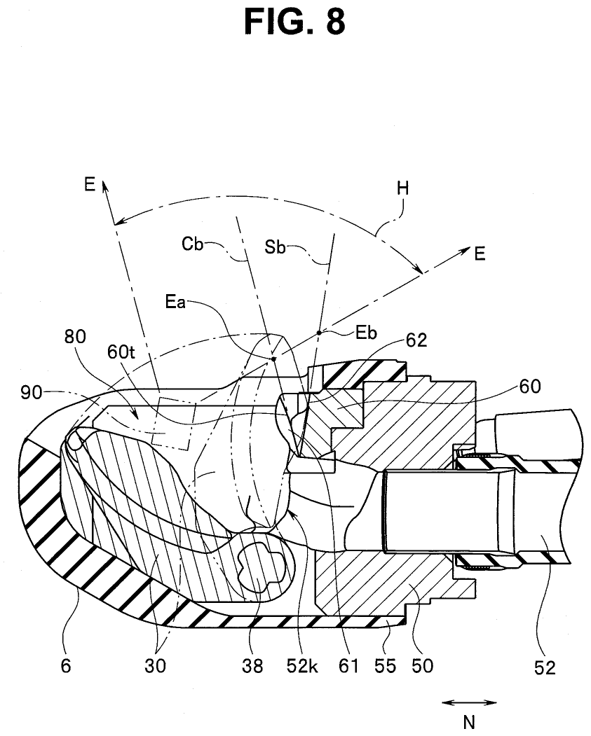

FIG. 6 is a top view of the distal end portion in FIG. 2 when seen in the VI direction in FIG. 2, FIG. 7 is a cross-sectional view of the distal end portion along the line VII-VII in FIG. 6, and FIG. 8 is a cross-sectional view of the distal end portion along the line VIII-VIII in FIG. 6.

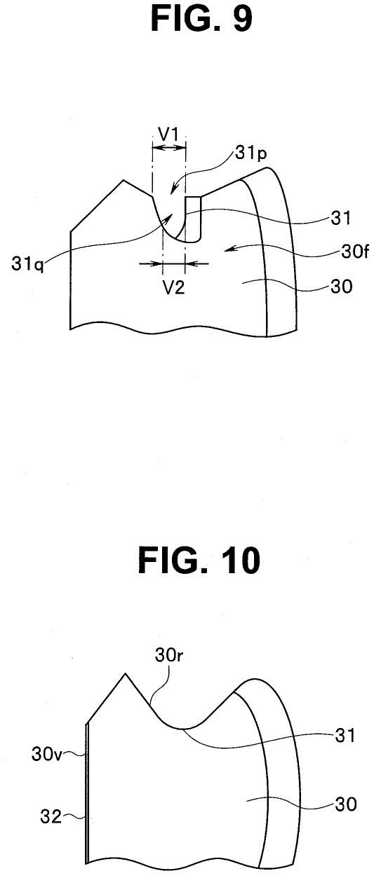

Moreover, FIG. 9 is a perspective view illustrating an example in which a first guide portion formed in a guide surface of the raising base in FIG. 2 is configured with a V-shaped groove, and FIG. 10 is a perspective view illustrating a modification in which the first guide portion of the raising base in FIG. 2 is formed at a ridge line of the other end of the raising base.

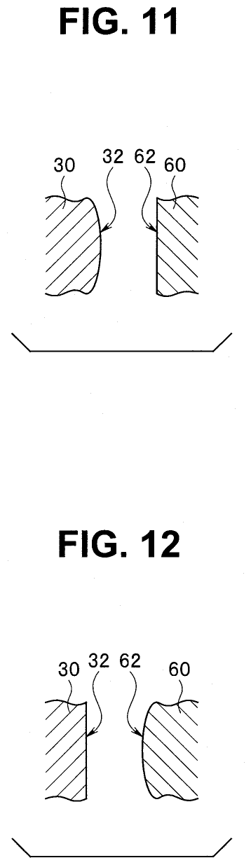

FIG. 11 is a partial cross-sectional view illustrating a modification in which a second guide portion of the raising base in FIG. 2 is formed into a curved surface and a second position of a guide wire abutting portion is formed into a plane, and FIG. 12 is a partial cross-sectional view illustrating a modification in which the second guide portion of the raising base in FIG. 2 is formed into a plane and the second position of the guide wire abutting portion is formed into a curved surface.

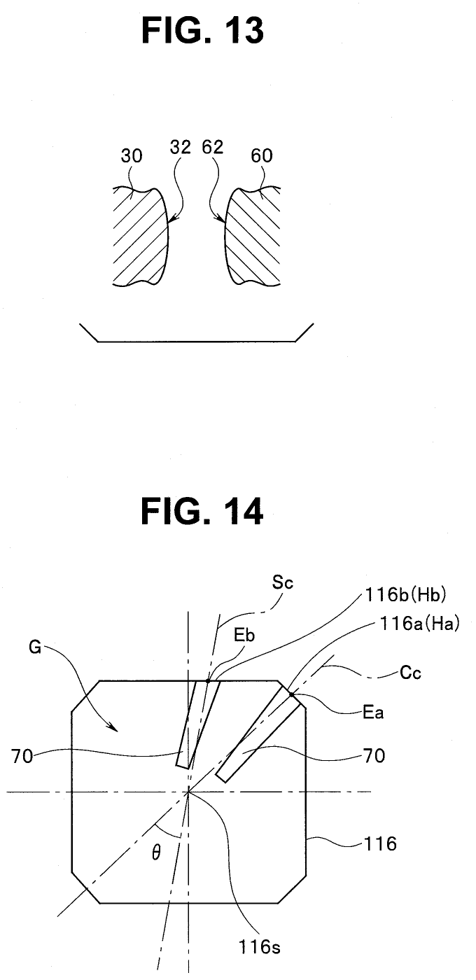

Moreover, FIG. 13 is a partial cross-sectional view illustrating a modification in which the second guide portion of the raising base in FIG. 2 is formed into a curved surface and the second position of the guide wire abutting portion is formed into a curved surface.

Furthermore, FIG. 14 is a diagram illustrating an example in which the guide wire appears in a monitor image displayed on a monitor in FIG. 1 in the case of the center locking and in the case of the side locking.

As illustrated in FIGS. 2 to 8, the distal end portion 6 includes a distal end rigid portion 50 made of metal, for example, and a distal end cover 55 formed of a non-conductive member such as a resin to cover the surroundings of the distal end rigid portion 50. The distal end cover 55 is fixed to the distal end rigid portion 50 with an adhesive or the like. Note that the distal end rigid portion 50 may be made of a resin.

A raising base accommodation portion 80 that has a side surface opening 80k at a part of an outer peripheral surface 6g of the distal end portion 6 and a part of an outer peripheral surface of the distal end rigid portion 50 is provided inside the distal end rigid portion 50.

An opening is provided in the raising base accommodation portion 80 such that a distal end of an insertion channel 52 inserted into the insertion portion 4, the operation portion 3, the universal cord 5, and the connector 12 faces the raising base accommodation portion 80 as illustrated in FIG. 8. Note that hereinafter, the distal end opening of the insertion channel 52 facing the raising base accommodation portion 80 will be referred to as an opening 52k.

An observation optical system 25 of an image pickup unit 90 or camera (see FIGS. 7 and 8) and an illumination lens 26 of an illumination optical system which are provided inside the distal end rigid portion 50 are provided at a position of the distal end rigid portion 50 at which a side surface opening 80k is formed. The image pickup unit 90 or camera can include lenses and an image sensor. In addition, it can include a lens barrel that holds the lenses and wiring for the image sensor. The position of the treatment instrument and the visual field range can be captured by the camera with the lenses and image sensor.

The image pickup unit 90 picks up an image of a site to be examined on a side on which the side surface opening 80k is formed, which is a side in the longitudinal axis direction N, via the observation optical system 25. The illumination lens 26 emits illumination light to the side in the longitudinal axis direction N. In other words, the endoscope 2 is a so-called side-viewing endoscope.

Also, the raising base 30 is provided in the raising base accommodation portion 80 at a position at which the raising base 30 faces the opening 52k.

The raising base 30 guides a treatment instrument that has passed inside the insertion channel 52 and has projected from the side surface opening 80k to a desired position by causing the treatment instrument to be raised and fixes the guide wire 70 that has passed inside the insertion channel 52 and has projected from the side surface opening 80k by causing the guide wire 70 to be raised.

Note that the guide wire 70 is typically configured to have an elastic force by covering a core line made of a super-elastic alloy with a flexible outer resin such as Teflon (registered trademark) or urethane (thermally contracting tube type), for example. In recent years, Teflon-coated guide wires have also been released.

The raising base 30 can be freely raised and lowered with rotation about a rotation shaft 38 provided on a side of one end of the raising base 30 located below the raising base accommodation portion 80 inside the raising base accommodation portion 80 by the rotation shaft 38 being axially supported to be freely rotatable relative to the distal end rigid portion 50, as illustrated in FIG. 8.

Note that a position at which the raising base 30 abuts on the guide wire 70 is a maximum raising position of the raising base 30 and a position at which a portion of the raising base 30 on the side of the other end abuts on a bottom surface of the raising base accommodation portion is a lowered position of the raising base 30 when the portion of the raising base 30 on the side of the other end and a guide wire abutting portion 60, which will be described later, pinch the guide wire 70.

One end of a raising wire inserted into the insertion portion 4 and the operation portion 3 is connected to the raising base 30. The other end of the raising wire is connected to a raising base drive mechanism provided inside the operation portion 3, which is not illustrated.

The raising base 30 has a configuration in which the raising base 30 is caused to stand up as illustrated in FIG. 3 from a state in which the raising base 30 is lowered as illustrated in FIG. 2 around the rotation shaft 38 or on the contrary, the raising base 30 is lowered from the raised state via the raising base drive mechanism, the raising wire, and the like by operating the treatment instrument raising base operation knob 20.

Also, as illustrated in FIG. 6, the surface of the raising base 30 facing the side surface opening 80k in the lowered state configures a guide surface 30f that guides the treatment instrument to the side surface opening 80k, and a first guide portion 31 is formed at a substantial center of the guide surface 30f at a position near the portion of the raising base 30 on the side of the other end.

The first guide portion 31 is configured with a groove portion formed into a recessed shape in the guide surface 30f. In one example, the first guide portion 31 has a minute portion 31q with a narrow width (V2) and an enlarged portion 31p with a wider width than the width of the minute portion (V1>V2) in a rotation shaft direction J of the rotation shaft 38 and is configured with a V-shaped or U-shaped groove, as illustrated in FIG. 9.

Note that the first guide portion 31 may be a recess provided at a ridge line 30r on the side of the other end instead of being configured with the groove formed in the guide surface 30f, as illustrated in FIG. 10.

The first guide portion 31 is used for the aforementioned center locking in which the guide wire 70 led out from the opening 52k toward the side surface opening 80k is pinched and fixed between the raising base 30 and a first position 61 of the guide wire abutting portion 60, which will be described later, as illustrated in FIG. 4 in a state in which the raising base 30 is raised, specifically, in a state in which the raising base 30 is raised to the maximum extent.

Moreover, the first guide portion 31 holds the treatment instrument at the guide surface 30f when the projecting direction of the treatment instrument from the side surface opening 80k is caused to vary with raising and lowering of the raising base 30.

Note that the center locking may be performed not only in the maximum raised state of the raising base 30 but also in an arbitrary raised state of the raising base 30.

Also, as illustrated in FIG. 6, a second guide portion 32 configured with a linear straight line portion in the longitudinal axis direction N is formed at a position separated from the first guide portion 31 of the guide surface 30f on the side of the observation optical system 25 in the rotation shaft direction J, for example, at an edge portion of the guide surface 30f.

A surface of the straight line portion of the second guide portion 32 on which the guide wire 70 is placed is configured with a plane. Note that the placement surface of the straight line portion is not limited to the plane and may be configured with an edge part 30v formed at the edge portion of the raising base 30 as illustrated in FIG. 10.

The second guide portion 32 is used for the aforementioned side locking in which the guide wire 70 led out from the opening 52k toward the side surface opening 80k is pinched and fixed between the raising base 30 and a second position 62 of the guide wire abutting portion 60, which will be described later, as illustrated in FIG. 5, in a state in which the raising base 30 is raised, specifically, in a state in which the raising base 30 is raised to a maximum extent.

Note that the side locking is not limited to the maximum raised state of the raising base 30 and may be performed in an arbitrary raised state of the raising base 30.

As illustrated in FIGS. 2 to 6 and 8, a surface of the distal end rigid portion 50 facing the raising base accommodation portion 80, specifically, a wall portion located at the proximal end of the side surface opening 80k is provided with the guide wire abutting portion 60 made of an insulating material, for example. Note that the guide wire abutting portion 60 may not be made of an insulating material.

The guide wire abutting portion 60 is a part on which the guide wire 70 led out from the opening 52k toward the side surface opening 80k abuts with the raising of the raising base 30 and is formed above the opening 52k in the distal end rigid portion 50.

As illustrated in FIG. 8, the guide wire abutting portion 60 has a projecting portion 60t projecting forward in the longitudinal axis direction N (hereinafter, simply referred to as forward) and facing the first guide portion 31 in a state in which the raising base 30 is raised to the maximum extent.

Moreover, the first position 61, to which the guide wire 70 is guided by the first guide portion 31 of the raising base 30, and at which the guide wire 70 is fixed between the first guide portion 31 and the guide wire abutting portion 60, is formed at a substantial center of the projecting portion 60t in the rotation shaft direction J.

Note that the first position 61 may be formed into a recessed shape to facilitate fitting of the guide wire 70.

In a case in which the center locking of the guide wire 70 is established with the first position 61 and the first guide portion 31, a method of pinching and fixing the guide wire 70 between the first position 61 and the first guide portion 31 and a method of fixing the guide wire 70 with the guide wire deviating in a shear manner using a reaction force of the guide wire 70 between the first position 61 and the first guide portion 31 are exemplified.

Also, the second position 62 is formed in the guide wire abutting portion 60 at a position which is not the projecting portion 60t and is separated from the first position 61 on the side of the observation optical system 25 in the rotation shaft direction J, specifically, the position that the second guide portion 32 faces when the raising base 30 is raised to the maximum extent.

The second position 62 is a position, to which the guide wire 70 is guided by the second guide portion 32 of the raising base 30, and at which the guide wire 70 is fixed between the second guide portion 32 and the guide wire abutting portion 60.

Note that in a case in which the side locking of the guide wire 70 is established with the second position 62 and the second guide portion 32, a method of pinching and fixing the guide wire 70 between the second position 62 and the second guide portion 32 is exemplified.

The second position 62 may be formed into a recessed shape to facilitate fitting of the guide wire 70 in the state in which the raising base 30 is raised or may be configured with an engagement groove with which the guide wire 70 is engaged.

Moreover, although the second position 62 and the second guide portion 32 face each other in parallel in FIG. 8, the second position 62 and the second guide portion 32 do not necessarily face each other in parallel.

Note that the description has been given hitherto on the assumption that the second guide portion 32 is configured with a plane and the second position 62 is configured with an engagement groove.

However, the second guide portion 32 may be configured with a curved surface, and the second position 62 may be configured with a plane, as illustrated in FIG. 11. As illustrated in FIG. 12, the second guide portion 32 may be configured with a plane, and the second position 62 may be configured with a curved surface. Moreover, as illustrated in FIG. 13, the second guide portion 32 may be configured with a curved surface, and the second position 62 may also be configured with a curved surface.

In any of the cases in FIGS. 11 to 13, it is possible to reliably perform the side locking of the guide wire 70 with the second guide portion 32 and the second position 62.

Not that it is necessary to set a clearance formed between the second guide portion 32 and the second position 62 to be smaller than the outer diameter of the guide wire 70 in the side locking state when the raising base 30 is caused to be raised to the maximum extent in order to reliably perform the side locking.

As illustrated in FIG. 3, a distance N2 from a bottom surface of the groove of the first guide portion 31 to the first position 61 is different from a distance N1 from a surface of the second guide portion 32 to the second position 62 in the longitudinal axis direction N (N1<N2) in the maximum raised state of the raising base 30.

Therefore, since the distance between the raising base 30 and the guide wire abutting portion 60 in the longitudinal axis direction N cannot be shortened at the time of the center locking than at the time of the side locking, fixing strength of the guide wire 70 is weaker than fixing strength achieved by the side locking.

Here, the second position 62 is located further backward than the first position 61 in the longitudinal axis direction N (hereinafter, simply referred to as backward) in the guide wire abutting portion 60 as illustrated in FIGS. 2 to 6 and 8.

Also, the second position 62 is located separately from the first position 61 in the rotation shaft direction J as described above. Moreover, the first guide portion 31 is formed to intersect the longitudinal axis direction N, and the second guide portion 32 is formed in the longitudinal axis direction N, in the guide surface 30f as illustrated in FIG. 6.

For this reason, a projecting (raising) position, a projecting (raising) angle, and a projecting direction of the guide wire 70 projecting in a visual field direction E of the image pickup unit 90 via the side surface opening 80k significantly differ in a case in which the center locking is performed and in a case in which the side locking is performed, as illustrated in FIGS. 4 and 5.

Therefore, a straight line Sa connecting the second guide portion 32 and a center 52c of the opening 52k of the insertion channel 52 when the second guide portion 32 performs the side locking of the guide wire 70 and a center axis Ca of the guide wire 70 when the first guide portion 31 performs the center locking of the guide wire 70 as illustrated by the two-dotted dashed line are formed in directions gradually separated from each other in the visual field direction E of the image pickup unit 90 in the state in which the raising base 30 is raised at the maximum angle as illustrated in FIG. 7.

As illustrated in FIG. 8, a straight line Sb including the straight line portion of the second guide portion 32 when the second guide portion 32 performs the side locking of the guide wire 70 and a center axis Cb of the guide wire 70 when the first guide portion 31 performs the center locking of the guide wire 70 as illustrated by the two-dotted dashed line are formed in directions gradually separated from each other in the visual field direction E of the image pickup unit 90 in the state in which the raising base 30 is raised at the maximum angle.

Therefore, in a case in which an image pickup range H of the image pickup unit 90 is defined relative to an image pickup center HC (see FIG. 7) as illustrated in FIG. 8, an intersection Ea of the guide wire 70 when the guide wire 70 is fixed through the center locking relative with the image pickup range H and an intersection Eb of the guide wire 70 when the guide wire 70 is fixed through the side locking are separated from each other by 1 mm or more, for example.

Note that it is assumed that the raising base 30 does not interfere with the image pickup range H both in the states in which the guide wire 70 is fixed through the center locking and through the side locking in the present embodiment.

However, there is a high probability that the raising base 30 interferes with the image pickup range H in a region Ha depending on setting of the maximum raising angle as illustrated in FIG. 3 in the center locking.

In order to prevent the interference, narrowing of the image pickup range H, size reduction of the raising base 30, an increase in distance between the observation optical system 25 and the raising base 30, and locating of the position where the observation optical system 25 is provided at a higher position in the visual field direction E in FIGS. 7 and 8, and the like are exemplified.

As a result, the center-locked guide wire 70 appears in a screen of the monitor image G displayed on the monitor 16, for example, at an edge 116 of an octagonal shape, to extend so as to intersect a first edge 116a corresponding to the region Ha by being located in the region Ha (see FIG. 3) in the image pickup range H as illustrated in FIG. 14. Also, the side-locked guide wire 70 appears to extend so as to intersect a second edge 116b that corresponds to a region Hb and is different from the first edge 116a by being located in the region Hb (see FIG. 3) in the image pickup range H.

At this time, an angle θ formed by a line segment Cc connecting a monitor center 116s and the intersection Ea and a line segment Sc connecting the monitor center 116s and the intersection Eb is 10° or more.

In other words, the first guide portion 31 guides the guide wire 70 to the first position 61 such that the guide wire 70 appears in the monitor image G with the guide wire 70 intersecting the first edge 116a by fixing the guide wire 70 at the first position 61 at the time of the center locking.

The second guide portion 32 guides the guide wire 70 to the second position 62 such that the guide wire 70 appears in the monitor image G at an angle different from the angle at the time of the center locking by 10° or more with the guide wire 70 intersecting the second edge 116b by fixing the guide wire 70 at the second position 62.

Note that the other configurations are similar to the configurations in an endoscope apparatus in the related art.

In this manner, the second position 62 is located further backward than the first position 61 in the longitudinal axis direction N (hereinafter, simply referred to as backward) and the second position 62 is located separately from the first position 61 in the rotation shaft direction J in the guide wire abutting portion 60, in the description of the present embodiment. Also, the guide wire 70 guided by the first guide portion 31 is located in the region Ha in the image pickup range H at the time of the center locking and the guide wire 70 guided by the second guide portion 32 is located in the region Hb in the image pickup range H at the time of the side locking, in the above description.

Moreover, the projecting (raising) position and the projecting (raising) angle of the guide wire 70 projecting in the visual field direction E of the image pickup unit 90 via the side surface opening 80k significantly differ as illustrated in FIGS. 4 and 5 in the case in which the center locking is performed and in the case in which the side locking is performed, in the above description.

Also, the first guide portion 31 guides the guide wire 70 to the first position 61 such that the guide wire 70 appears in the monitor image G with the guide wire 70 intersecting the first edge 116a by fixing the guide wire 70 at the first position 61 and causing the guide wire 70 to be located in the region Ha at the time of the center locking, in the above description.

Moreover, the second guide portion 32 guides the guide wire 70 to the second position 62 such that the guide wire 70 appears in the monitor image G with the guide wire 70 intersecting the second edge 116b by fixing the guide wire 70 at the second position 62 and causing the guide wire 70 to be located in the region Hb at the time of the side locking, in the above description.

As a result, at the edges 116 of the monitor image G displayed on the monitor 16, the center-locked guide wire 70 appears to extend so as to intersect the first edge 116a corresponding to the region Ha (see FIG. 3) in the image pickup range H, and the side-locked guide wire 70 appears to extend so as to intersect the second edge 116b that corresponds to the region Hb (see FIG. 3) in the image pickup range H and is different from the first edge 116a.

Therefore, a surgeon who observes the monitor image G on the monitor 16 can easily recognize that the guide wire 70 is being center-locked in the case in which the guide wire 70 appears to extend in an intersecting manner from the first edge 116a of the edges 116 when only one guide wire 70 appears in the monitor image G. Also, the surgeon can easily recognize that the guide wire 70 is being side-locked in the case in which the guide wire 70 appears to extend in an intersecting manner from the second edge 116b.

In other words, the surgeon can easily identify which of the center locking and the side locking is being performed, by the guide wire 70 extending from the different edges.

As described above, it is possible to provide the endoscope apparatus 1 with a configuration that enables the surgeon to easily identify which of the center locking and the side locking is being performed when the guide wire 70 is fixed with the guide wire abutting portion 60 with the raising of the raising base 30.

### Second Embodiment

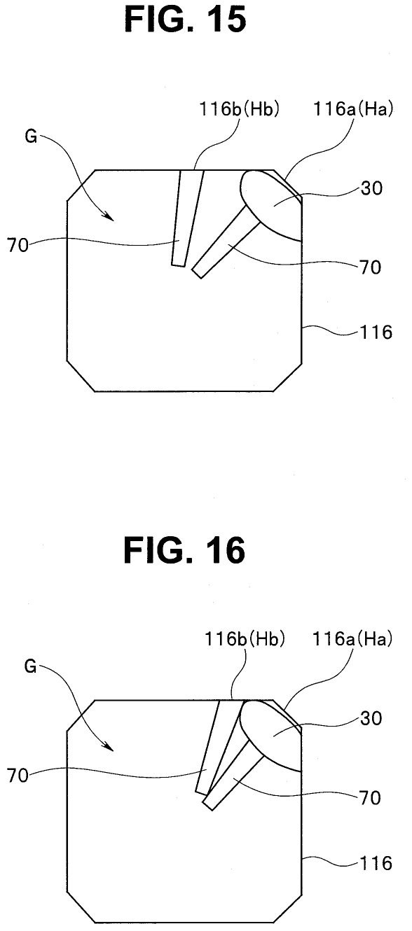

FIG. 15 is a diagram illustrating a configuration in which a raising base appears in a monitor image displayed on a monitor in an endoscope apparatus according to the present embodiment.

A configuration of the endoscope apparatus according to the second embodiment is different from the configuration of the endoscope apparatus according to the first embodiment illustrated in FIGS. 1 to 14 as described above in that a raising base appears in a monitor image at the time of center locking.

Therefore, only the difference will be described, the same reference signs will be applied to components similar to the components in the first embodiment, and the description will be omitted.

As illustrated in FIG. 15, a raising base 30 appears in a monitor image G at the time of center locking in the present embodiment.

Specifically, the raising base 30 interferes with an image pickup range H in the center locking state as illustrated in FIG. 3.

Moreover, only a first guide portion 31 is disposed within the image pickup range H, and a second guide portion 32 is disposed outside the image pickup range H, in the center locking state.

Note that the first guide portion 31 may interfere with a range Ha in the image pickup range H similarly to the first embodiment.

Also, an interference range of the raising base 30 with the image pickup range H at the time of the center locking is preferably further backward than a center position Ht of the image pickup range H in parallel with the rotation shaft direction J, that is, on the side of a guide wire abutting portion 60 as illustrated in FIG. 3.

This is because the surgeon can more easily identify which of the center locking and the side locking is being performed when the raising angle of the raising base 30 is larger since the guide wire 70 as a target of the identification appears in a wider range in the monitor image G.

With such a configuration, the guide wire 70 appears to extend so as to intersect the raising base 30 appearing in the monitor image G when the center locking is performed as illustrated in FIG. 15, and the guide wire 70 appears to extend so as to intersect a second edge 116b similarly to the first embodiment when the side locking is performed.

Similarly to the first embodiment, the angle of the guide wire 70 appearing in the monitor image G differs at the time of the center locking and at the time of the side locking.

In this manner, the surgeon can more easily identify which of the center locking and the side locking is being performed than in the first embodiment, depending on the angle of the guide wire 70 appearing in the monitor image G and which of the raising base 30 and the second edge 116b the guide wire extends from.

Moreover, the surgeon can easily identify which of the center locking and the side locking is being performed since the guide wire 70 appears in mutually separated locations, that is, the guide wire 70 appears to extend so as to intersect the first edge 116a and the raising base 30 at the time of the center locking and appears to extend so as to intersect the second edge 116b at the time of the side locking similarly to the first embodiment in a case in which the first guide portion 31 interferes with the region Ha in the image pickup range H similarly to the first embodiment.

Note that the other advantages are the same as the advantages of the aforementioned first embodiment.

Hereinafter, a modification will be described using FIG. 16. FIG. 16 is a diagram illustrating a modification in which guide wires are displayed in further proximity to each other in monitor images in a case of center locking and in a case of side locking as compared with FIG. 15.

As illustrated in FIG. 16, the surgeon can easily identify which of the center locking and the side locking is being performed on the basis of whether or not the guide wire appears to intersect the raising base 30 similarly to the present embodiment even if the guide wire 70 is displayed at proximal positions in the monitor image G in the center locking and in the side locking, as illustrated in FIG. 16. Note that the other advantages are the same as the advantages of the aforementioned present embodiment.

Moreover, although the aforementioned first and second embodiments have been described on the assumption that the guide wire abutting portion 60 is formed at the distal end rigid portion 50, the present invention is not limited thereto, and the guide wire abutting portion 60 may be formed separately from the distal end rigid portion 50.

Although the graphical user interface has been described by exemplifying the screen of the monitor 16, the present invention is not limited thereto, and the graphical user interface may be image data reduced in size.

In other words, the edges 116a and 116b of the screen on which the monitor image G is displayed may be edges of the image data reduced in size.

Moreover, although the edges 116 of the screen on which the monitor image G is displayed form an octagonal shape in the above description, the present invention is not limited thereto, and it is a matter of course that the shape is not limited to the octagonal shape and may be any shape as long as the shape has a plurality of intersecting edges.

What is claimed is:

1. An endoscope apparatus comprising:
a distal end portion provided on a side of a distal end of an insertion portion in a longitudinal axis direction;
a raising base accommodation portion provided inside the distal end portion, the raising base accommodation portion including a side surface opening provided at a part of an outer peripheral surface of the distal end portion;
an insertion channel provided inside the insertion portion and including an opening at a distal end in the longitudinal axis direction and positioned to face the raising base accommodation portion;
a camera provided in the distal end portion and configured to image a site to be examined on a side on which the side surface opening is formed;
a raising base provided in the raising base accommodation portion to face the opening of the insertion channel and configured to be freely raised and lowered;
a guide wire abutting portion provided inside the distal end portion and configured to abut a guide wire that is accommodated by the insertion channel when the raising base is raised;
a first guide portion provided in the raising base and configured to guide the guide wire accommodated by the insertion channel to fix the guide wire at a first position of the guide wire abutting portion when the raising base is raised; and
a second guide portion provided in the raising base spaced apart from the first guide portion in a rotation shaft direction of the raising base and configured to guide the guide wire accommodated by the insertion channel to fix the guide wire at a second position of the guide wire abutting portion when the raising base is raised, wherein:
with respect to edges of the image of the site, the first guide portion is configured to guide the guide wire to the first position such that the guide wire appears in the image with the guide wire intersecting a first edge, and
the second guide portion is configured to guide the guide wire to the second position such that the guide wire appears in the image with the guide wire intersecting a second edge that is different from the first edge.

2. An endoscope apparatus comprising:
a distal end portion provided on a side of a distal end of an insertion portion in a longitudinal axis direction;
a raising base accommodation portion provided inside the distal end portion, the raising base accommodation portion including a side surface opening at a part of an outer peripheral surface of the distal end portion;
an insertion channel provided inside the insertion portion and including an opening at a distal end in the longitudinal axis direction positioned to face the raising base accommodation portion;
a camera provided in the distal end portion and configured to image a site to be examined on a side on which the side surface opening is formed;
a raising base provided in the raising base accommodation portion to face the opening of the insertion channel and configured to be freely raised and lowered;
a guide wire abutting portion provided inside the distal end portion and configured to abut a guide wire accommodated by the insertion channel by raising of the raising base;
a first guide portion provided in the raising base and configured to guide the guide wire accommodated by the insertion channel to fix the guide wire at a first position of the guide wire abutting portion when the raising base is raised; and
a second guide portion provided in the raising base spaced apart from the first guide portion in a rotation shaft direction of the raising base and configured to guide the guide wire accommodated by the insertion channel to fix the guide wire at a second position of the guide wire abutting portion when the raising base is raised,
wherein when the raising base is raised, the first guide portion is disposed within an image pickup range of the camera, and the second guide portion is disposed outside the image pickup range.

3. The endoscope apparatus according to claim 1, wherein:
when the raising base is raised, a straight line connecting a center of the opening of the insertion channel to the second guide portion when the second guide portion fixes the guide wire is formed in a first direction, and a center axis of the guide wire when the first guide portion fixes the guide wire is formed a second direction, and
the first direction gradually diverges from the second direction in a visual field direction of the camera.

4. The endoscope apparatus according to claim 1, wherein:
the second guide portion includes a straight line portion provided at an edge of the raising base in the longitudinal axis direction,
when the raising base is raised, a straight line including the straight line portion, when the second guide portion fixes the guide wire extends along a first direction, and a center axis of the guide wire, when the first guide portion fixes the guide wire, extends along a second direction, and
the first direction gradually diverges from the second direction in a visual field direction of the camera.

5. The endoscope apparatus according to claim 1, wherein an engagement groove configured to engage with the guide wire when the raising base is raised is provided at the second position of the guide wire abutting portion.

6. The endoscope apparatus according to claim 1, wherein the first guide portion is a groove portion provided in the raising base.

7. The endoscope apparatus according to claim 1, wherein the first guide portion includes:
a narrow portion having a first width in the rotation shaft direction, and
a wide portion having a second width that is wider than the first width in the rotation shaft direction.

8. The endoscope apparatus according to claim 1, wherein the guide wire abutting portion includes a projecting portion configured to project in the longitudinal axis direction and to face the first guide portion when the raising base is raised.

9. The endoscope apparatus according to claim 1, wherein the guide wire abutting portion is provided above the opening of the insertion channel at a distal end rigid portion in the distal end portion.

10. The endoscope apparatus according to claim 9, wherein
the first guide portion is a groove portion provided in the raising base, and the second guide portion is a side edge of the raising base, and
the first position on which the groove portion abuts and the second position on which the side edge abuts are provided in the distal end rigid portion.

11. The endoscope apparatus according to claim 1, wherein when the raising base is raised, the first guide portion is disposed in an image pickup range of the camera.

12. An endoscope apparatus comprising:
a distal end portion provided on a side of a distal end of an insertion portion in a longitudinal axis direction;
a raising base accommodation portion provided inside the distal end portion, the raising base accommodation portion including a side surface opening at a part of an outer peripheral surface of the distal end portion;
an insertion channel provided inside the insertion portion and including an opening at a distal end in the longitudinal axis direction positioned to face the raising base accommodation portion;
camera provided in the distal end portion and configured to image a site a side on which the side surface opening is formed;
a raising base provided in the raising base accommodation portion positioned to face the opening of the insertion channel and configured to be freely raised and lowered;
a first guide portion provided in the raising base and configured to be able to guide, to a first position, a treatment instrument accommodated by the opening of the insertion channel; and
a second guide portion provided in the raising base at a position different from a position of the first guide portion and configured to be able to guide, to a second position, the treatment instrument accommodated by the opening of the insertion channel,
wherein:
when the raising base is rotated and is in a first state, with respect to edges of the image of the site picked up by the camera, the first guide portion guides the treatment instrument to the first position such that the treatment instrument appears in the image with the treatment instrument intersecting a first edge, and
the second guide portion guides the treatment instrument to the second position such that the treatment instrument appears in the image with the treatment instrument intersecting a second edge that is different from the first edge.

13. The endoscope apparatus of claim 1, wherein the raising base is raised and lowered due to rotation of the raising base about a rotation shaft that extends through the raising base.

14. The endoscope apparatus of claim 2, wherein the raising base is raised and lowered due to rotation of the raising base about a rotation shaft that extends through the raising base.

15. The endoscope apparatus of claim 12, wherein the raising base is raised and lowered due to rotation of the raising base about a rotation shaft that extends through the raising base.
